(19) United States
(12) Patent Application Publication
(10) Pub. No.: US 2023/0238118 A1
(43) Pub. Date: Jul. 27, 2023

(54) INFORMATION PROCESSING APPARATUS, INFORMATION PROCESSING SYSTEM, INFORMATION PROCESSING METHOD, AND PROGRAM

(71) Applicant: FUJIFILM Corporation, Tokyo (JP)

(72) Inventor: Keigo NAKAMURA, Tokyo (JP)

(73) Assignee: FUJIFILM Corporation, Tokyo (JP)

(21) Appl. No.: 18/069,991

(22) Filed: Dec. 21, 2022

(30) Foreign Application Priority Data
Jan. 24, 2022 (JP) 2022-008921

(51) Int. Cl. G16H 30/40; G06V 10/62; G06T 7/11; G06T 11/20; G06V 10/25; G16H 15/00

(52) U.S. Cl. CPC G16H 30/40; G06V 10/62; G06T 7/11; G06T 11/206; G06V 10/25; G16H 15/00; G06T 2207/30096

(57) ABSTRACT

There are provided an information processing apparatus, an information processing system, an information processing method, and a program which can intuitively grasp a state of a region of interest.

An information processing apparatus includes at least one processor; and at least one memory that stores a command for the at least one processor to execute, in which the at least one processor acquires an analysis result relating to a characteristic of a region of interest of an image, and generates a diagram on the basis of the analysis result. The diagram expresses a state of the region of interest having an exclusive relationship by at least one of a spread direction or an area.

### CROSS-REFERENCE TO RELATED APPLICATION

The present application claims priority under 35 U.S.0 §119(a) to Japanese Patent Application No. 2022-008921 filed on Jan. 24, 2022, which is hereby expressly incorporated by reference, in its entirety, into the present application.

### BACKGROUND OF THE INVENTION

### 1. Field of the Invention

The present invention relates to an information processing apparatus, an information processing system, an information processing method, and a program, and particularly relates to a technique of providing an analysis result of an image.

### 2. Description of the Related Art

A radiologist in the radiology department diagnoses medical images in response to an image diagnosis request from the attending physician of the medical department, and creates an interpretation report describing the presence or absence of abnormalities. A technique has been known for supporting the creation of a findings sentence in a case of creating such an interpretation report. For example, word candidates to be input to the findings sentence are displayed from structured data.

In recent years, in the medical institution, the contents described in the interpretation report are not properly delivered to the attending physician so that the treatment intervention is delayed, which has become a problem. It is said that there are a plurality of causes for this, but one of the causes is that the interpretation report is composed only of characters and is not intuitive.

For such a problem, JP2019-118820A discloses a technique of generating and displaying a radar chart of a part of interest of a subject.

### SUMMARY OF THE INVENTION

However, there is a problem that it is difficult to grasp the state of a lesion simply by displaying the radar chart of quantitative values.

The present invention is made in view of such circumstances, and an object thereof is to provide an information processing apparatus, an information processing system, an information processing method, and a program which can intuitively grasp a state of a region of interest.

An aspect of an information processing apparatus for achieving the object is an information processing apparatus comprising at least one processor; and at least one memory that stores a command for the at least one processor to execute, in which the at least one processor is configured to acquire an analysis result relating to a characteristic of a region of interest of an image, and generate a diagram on the basis of the analysis result, and the diagram expresses a state of the region of interest having an exclusive relationship by at least one of a spread direction or an area. According to the aspect, it is possible for the user to intuitively grasp the state of the region of interest.

It is preferable that the image is a medical image in which a subject is imaged. It is preferable that the state of the region of interest is benign and malignant. The state of the region of interest may be a primary cancer and a metastatic cancer, or may be a disease name. Thereby, it is possible for the user to grasp the state of the region of interest, which has an exclusive relationship, of the medical image, and to support the diagnosis of the medical image.

It is preferable that the at least one processor displays the diagram on a display. Thereby, it is possible for the user to grasp at a glance the state of the region of interest.

It is preferable that the at least one processor is configured to estimate the state of the region of interest, and display an estimated result on a display. Thereby, it is possible for the user to know the state of the region of interest.

It is preferable that the at least one processor estimates the state of the region of interest on the basis of at least one of the direction or the area of the diagram. Thereby, it is possible to accurately estimate the state of the region of interest.

It is preferable that the diagram includes a radar chart. Thereby, the state of the region of interest can be expressed to be easily understood.

It is preferable that the at least one processor acquires the analysis results of a plurality of images of the same subject, the plurality of images being captured at different times. It is preferable that the diagram expresses the analysis results of the plurality of images in a superimposed manner. It is preferable that the diagram expresses the analysis results of the plurality of images in different colors. Thereby, it is possible to express time-series changes of the state of the region of interest.

It is preferable that the at least one processor acquires the analysis results of a plurality of images, for which at least one of an imaging condition or an imaging device is different, of the same subject. Thereby, it is possible to express the differences in the state of the region of interest due to the differences in imaging conditions and imaging devices.

It is preferable that the imaging condition includes a contrast condition. Thereby, it is possible to express the differences in the state of the region of interest due to the difference in contrast conditions.

It is preferable that the at least one processor acquires the analysis result by analyzing the image. The at least one processor may acquire the analysis result by analyzing the interpretation report of the image. Thereby, it is possible to appropriately acquire the analysis result.

It is preferable that the at least one processor generates a diagram in which the analysis result acquired by analyzing the interpretation report and the analysis result acquired by analyzing the image are combined. Thereby, it is possible to more accurately express the state of the region of interest.

It is preferable that the at least one processor generates a diagram in which the analysis result acquired by analyzing the interpretation report and the analysis result acquired by analyzing the image are distinguished from each other. Thereby, it is possible to respectively express the state of the region of interest of the image and the state of the region of interest of the interpretation report.

It is preferable that the state of the region of interest having the exclusive relationship includes a first state, and a second state different from the first state, and in the diagram, an axis of a characteristic indicating a feature of the first state, and an axis of a characteristic indicating a feature of the second state are arranged on opposite sides from each other with respect to a center of the diagram. For example, in the diagram, the axis of the characteristic indicating the feature of the first state is arranged on the left side of the center of the diagram, and the axis of the characteristic indicating the feature of the second state is arranged on the right side of the center of the diagram. In the diagram, the axis of the characteristic indicating the feature of the first state may be arranged on the upper side of the center of the diagram, and the axis of the characteristic indicating the feature of the second state may be arranged on the lower side of the center of the diagram. Thereby, it is possible to express the state of the region of interest by the spread direction of the plotted dot of each axis.

It is preferable that a value of the axis is set to a relatively larger value as being away from the center of the diagram, and the at least one processor is configured to acquire the analysis result relating to the characteristic indicating the feature of the first state as a numerical value which is relatively larger as the characteristic more corresponds to the feature of the first state, and acquire the analysis result relating to the characteristic indicating the feature of the second state as a numerical value which is relatively larger as the characteristic more corresponds to the feature of the second state. Contrary, a value of the axis may be set to a relatively smaller value as being away from the center of the diagram, and the at least one processor may acquire the analysis result relating to the characteristic indicating the feature of the first state as a numerical value which is relatively smaller as the characteristic more corresponds to the feature of the first state, and acquire the analysis result relating to the characteristic indicating the feature of the second state as a numerical value which is relatively smaller as the characteristic more corresponds to the feature of the second state. Thereby, it is possible to express the state of the region of interest by the area of the region surrounded by the lines connecting the plotted dots of the axes.

An aspect of an information processing system for achieving the object is an information processing system comprising the information processing apparatus described above; an imaging device that captures the image; and a display on which the diagram is displayed. According to the aspect, it is possible for the user to intuitively grasp the state of the region of interest of the image captured by the imaging device, by the user visually checking the diagram displayed on the display.

An aspect of an information processing method for achieving the object is an information processing method comprising an analysis result acquisition step of acquiring an analysis result relating to a characteristic of a region of interest of an image; and a diagram generation step of generating a diagram on the basis of the analysis result, in which the diagram expresses a state of the region of interest having an exclusive relationship by at least one of a spread direction or an area. According to the aspect, it is possible for the user to intuitively grasp the state of the region of interest.

An aspect of a program for achieving the object is a program for causing a computer to execute the information processing method described above. According to the aspect, it is possible for the user to intuitively grasp the state of the region of interest. A computer-readable non-transitory storage medium in which the program is stored may be included in the aspect.

According to the present invention, it is possible to intuitively grasp the state of the region of interest.

### BRIEF DESCRIPTION OF THE DRAWINGS

FIG. 1 is an entire configuration diagram of a medical information processing system.

FIG. 2 is a block diagram illustrating a configuration of a medical information processing apparatus.

FIG. 3 is a flowchart illustrating a medical information processing method.

FIG. 4 is a diagram illustrating an example of a radar chart relating to a lung nodule.

FIG. 5 is a diagram illustrating an example of a medical image and a radar chart of a lung nodule corresponding to the medical image.

FIG. 6 is a diagram illustrating an example of a medical image and a radar chart of a lung nodule corresponding to the medical image.

FIG. 7 is a diagram illustrating an example of a radar chart relating to a liver tumor.

FIG. 8 is a table illustrating an example of a score of each item of an analysis result relating to characteristics of a liver tumor of a medical image.

FIG. 9 is a diagram illustrating an example of a medical image and a radar chart corresponding to the medical image.

FIG. 10 is a table illustrating an example of a score of each item of an analysis result relating to characteristics of a liver tumor of a medical image.

FIG. 11 is a diagram illustrating an example of a medical image and a radar chart corresponding to the medical image.

FIG. 12 is a table illustrating an example of a score of each item of an analysis result relating to characteristics of a liver tumor of a medical image.

FIG. 13 is a diagram illustrating an example of a medical image and a radar chart corresponding to the medical image.

FIG. 14 is a diagram illustrating an example of a radar chart displayed in color.

FIG. 15 is a diagram illustrating an example of a radar chart collectively expressing a plurality of lesions.

FIG. 16 is a diagram illustrating an example of a current medical image, a past image, and a radar chart corresponding to each of the medical images.

FIG. 17 is a diagram illustrating an example of contents of an interpretation report of a medical image.

FIG. 18 is a diagram illustrating an interpretation report attached to a radar chart.

### DESCRIPTION OF THE PREFERRED EMBODIMENTS

Hereinafter, preferred embodiments of the present invention will be described in detail with reference to the accompanying drawings.

### Medical Image Processing System

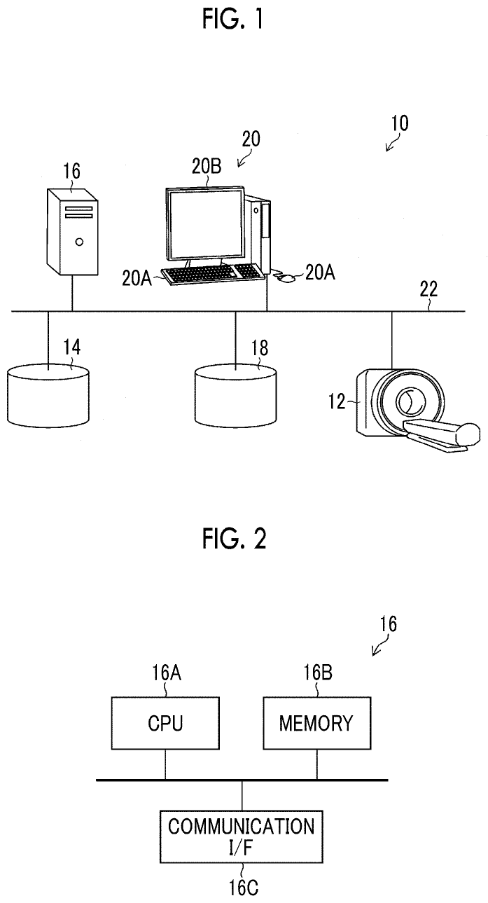

A medical information processing system according to the present embodiment is a system which captures a medical image of a subject (patient), performs characteristic analysis of a lesion in the captured medical image, generates a diagram on the basis of a result of the characteristic analysis, and presents the generated diagram. FIG. 1 is an entire configuration diagram of a medical information processing system 10. As illustrated in FIG. 1, the medical information processing system 10 includes a medical image examination device 12, a medical image database 14, a medical information processing apparatus 16, an interpretation report database 18, and a user terminal 20.

The medical image examination device 12, the medical image database 14, the medical information processing apparatus 16, the interpretation report database 18, and the user terminal 20 are connected via a network 22 to transmit and receive data to and from each other. The network 22 includes a wired or wireless local area network (LAN) for communication connection of various devices in the medical institution. The network 22 may include a wide area network (WAN) that connects LANs of a plurality of medical institutions.

The medical image examination device 12 is an imaging device that images an examination target part of an examination target and generates a medical image. Examples of the medical image examination device 12 include an X-ray imaging device, a computed tomography (CT) device, a magnetic resonance imaging (MRI) device, a positron emission tomography (PET) device, an ultrasound device, and a computed radiography (CR) device using flat X-ray detector.

The medical image database 14 is a database that manages the medical image captured by the medical image examination device 12. As the medical image database 14, a computer including a large-capacity storage device for storing the medical image is applied. Software providing a function of a database management system is incorporated in the computer.

As a format of the medical image, Digital Imaging and Communications in Medicine (Dicom) standards can be applied. The medical image may be added with accessory information (Dicom tag information) defined in the Dicom standards. The term “image” used in this specification includes not only the image itself, such as a photograph, but also image data which is a signal representing an image.

The medical information processing apparatus 16 analyzes the medical image, and acquires an analysis result relating to the characteristics of the region of interest in the medical image. The medical information processing apparatus 16 may analyze the interpretation report of the medical image, and acquire an analysis result relating to the characteristics of the region of interest. Further, the medical information processing apparatus 16 generates a diagram on the basis of the analysis result. The medical information processing apparatus 16 may automatically generate the interpretation report of the medical image.

As the medical information processing apparatus 16, a personal computer or a workstation (an example of a “computer”) can be applied. FIG. 2 is a block diagram illustrating a configuration of the medical information processing apparatus 16. As illustrated in FIG. 2, the medical information processing apparatus 16 includes a processor 16A, a memory 16B, and a communication interface 16C.

The processor 16A executes a command stored in the memory 16B. The hardware structures of the processor 16A are the following various processors. The various processors include a central processing unit (CPU) as a general-purpose processor executing software (program) and acting as various functional units, a graphics processing unit (GPU) as a processor specialized for image processing, a programmable logic device (PLD) as a processor of which the circuit configuration can be changed after manufacturing such as a field programmable gate array (FPGA), a dedicated electrical circuit or the like as a processor having a circuit configuration designed exclusively for executing specific processing such as an application specific integrated circuit (ASIC).

One processing unit may be configured by one of the various processors, or configured by the same or different kinds of two or more processors (for example, combination of a plurality of FPGAs, combination of the CPU and the FPGA, combination of the CPU and the GPU, or the like). In addition, a plurality of functional units may be configured by one processor. As an example where a plurality of functional units are formed by one processor, first, there is an aspect where one processor is formed of a combination of one or more CPUs and software as typified by a computer, such as a client or a server, and this processor acts as a plurality of functional units. Second, there is a form where a processor fulfilling the functions of the entire system including a plurality of functional units by one integrated circuit (IC) chip as typified by a system on chip (SoC) or the like is used. In this manner, various functional units are configured by using one or more of the above-described various processors as hardware structures.

Furthermore, the hardware structures of these various processors are more specifically electrical circuitry where circuit elements, such as semiconductor elements, are combined.

The memory 16B stores a command for the processor 16A to execute. The memory 16B includes a random access memory (RAM) and a read only memory (ROM) (not illustrated). The processor 16A uses the RAM as a work area, executes software using various parameters and programs including an information processing program described later, which are stored in the ROM, and executes various kinds of processing of the medical information processing apparatus 16 by using the parameters stored in the ROM or the like.

The communication interface 16C controls communication with the medical image examination device 12, the medical image database 14, the interpretation report database 18, and the user terminal 20 via the network 22 according to a predetermined protocol.

The medical information processing apparatus 16 may be a cloud server that can be accessed from a plurality of medical institutions via the Internet. The processing performed in the medical information processing apparatus 16 may be a billing or fixed fee cloud service.

Returning to the description of FIG. 1, the interpretation report database 18 is a database that manages an interpretation report automatically generated by the medical information processing apparatus 16, and an interpretation report generated by a user such as a radiologist in the user terminal 20. As the interpretation report database 18, a computer including a large-capacity storage device for storing the interpretation report is applied. Software providing a function of a database management system is incorporated in the computer. The medical image database 14 and the interpretation report database 18 may be configured by one computer.

The user terminal 20 is a terminal device for the user to view and edit the interpretation report. As the user terminal 20, for example, a personal computer is applied. The user terminal 20 may be a workstation, or may be a tablet terminal. The user terminal 20 includes an input device 20A and a display 20B. The user inputs an instruction to the medical information processing system 10 by using the input device 20A. Further, the user terminal 20 displays the medical image and the interpretation report on the display 20B. The medical information processing apparatus 16 displays a diagram described later on the display 20B.

### Medical Information Processing Method

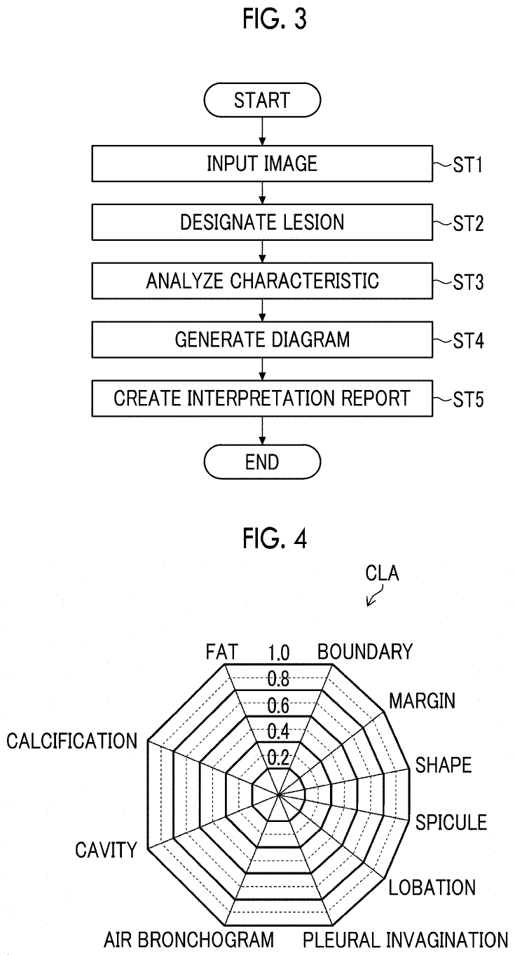

FIG. 3 is a flowchart illustrating a medical information processing method using the medical information processing system 10. Here, an example of creating an interpretation report for the medical image of the examination target will be described.

In an image input step in Step ST1, the medical image which is captured by the medical image examination device 12 and is stored in the medical image database 14 is transmitted (input) to the user terminal 20 operated by the user. The user terminal 20 receives the medical image, and displays the received medical image on the display 20B.

In a lesion designation step in Step ST2, a lesion (an example of the “region of interest”) is designated for the medical image input in the image input step. The designation of the lesion is performed by the user using the input device 20A while viewing the medical image displayed on the display 20B. As the designation of the lesion, the medical image may be automatically analyzed and designated by the computer-aided diagnosis (CAD) in the medical information processing apparatus 16 or the user terminal 20. Further, the designation of the lesion may be performed such that the medical information processing apparatus 16 or the user terminal 20 displays candidates of the region of the lesion automatically extracted by the CAD on the display 20B and the user performs selection among the displayed candidates using the input device 20A.

In a characteristic analysis step (an example of an “analysis result acquisition step”) in Step ST3, the medical information processing apparatus 16 performs a predetermined characteristic analysis on the lesion designated in the lesion designation step. The characteristic analysis may be identified by a method using deep learning. For example, it is possible to use a method described in Characterization of Pulmonary Nodules in Computed Tomography Images Based on Pseudo-Labeling using Radiology Reports.

The characteristic analysis result (an example of an “analysis result”) uses, for example, values from 0 to 1, and indicates that the higher the score, the more significant the findings for the item. The range of the score values may be a range of other numerical values, for example, 0 to 100. The characteristic analysis result may be output as discrete values. For example, values of four levels of “clear”, “slightly clear”, “slightly unclear”, and “unclear” may be used.

In a diagram generation step in Step ST4, the medical information processing apparatus 16 generates a diagram from the analysis result of the characteristic analysis step. In the present embodiment, the diagram expresses the state of the lesion having a mutually exclusive relationship by at least one of a spread direction or an area. The diagram includes a radar chart, a polygonal line graph, a bar graph, and the like. The radar chart is a graph in which, with respect to the radar chart coordinates in which a plurality of axes corresponding to a plurality of items are arranged radially around the origin, the score of the item corresponding to each axis is plotted, and the plotted point is connected to the plotted point on the adjacent axis by a straight line to form a polygonal graph.

In an interpretation report creation step in Step ST5, an interpretation report of the image input in the image input step is created. The interpretation report may be automatically generated by the medical information processing apparatus 16 as described above, and may be generated by the user in the user terminal 20. Further, the medical information processing apparatus 16 attaches the diagram created in the diagram generation step to the interpretation report, and stores the interpretation report in the interpretation report database 18.

The steps in Step ST3 to Step ST5 executed by the medical information processing apparatus 16 constitute the information processing method. The information processing method is realized by the processor 16A executing the information processing program stored in the memory 16B. The information processing program may be provided by a computer-readable non-transitory storage medium. In this case, the medical information processing apparatus 16 may read the information processing program from the non-transitory storage medium, and store the information processing program in the memory 16B.

### Radar Chart

### Lung Nodule

The radar chart relating to the lung nodule will be described. The state of the lung nodule includes benign and malignant which have a mutually exclusive relationship. Here, the radar chart will be described which expresses that the larger the area on the left side, the more benign, and the larger the area on the right side, the more malignant.

FIG. 4 is a diagram illustrating an example of radar chart coordinates CLA relating to the lung nodule. As illustrated in FIG. 4, the radar chart coordinates CLA has a plurality axes arranged radially around the origin, which respectively correspond to “boundary”, “margin”, “shape”, “spicule”, “lobation”, “pleural invagination”, “air bronchogram”, “cavity”, “calcification”, and “fat” as 10 items relating to the characteristics of the lesion of the lung nodule. The axis of each item is assigned a larger value as it moves away from the origin. The radar chart coordinates CLA has scale lines as solid straight lines and auxiliary lines as dashed straight lines, each of which connects the same value on each adjacent axis.

Here, since the number of items is 10, the shape of the radar chart coordinates CLA is a decagon. Note that the items relating to the characteristics of the lesion of the lung nodule are not limited to the 10 items described here. In a case where the number of items is N, which is an integer, the shape of the radar chart coordinates CLA is an N-polygon.

In the radar chart coordinates CLA, the axes of four items of “fat”, “calcification”, “cavity”, and “air bronchogram” which are benign features are arranged on the left side of the origin in FIG. 4, and the axes of six items of “boundary”, “margin”, “shape”, “spicule”, “lobation”, and “pleural invagination” which are malignant features are arranged on the right side of the origin in FIG. 4.

In the radar chart coordinates CLA, scores of each item are plotted on each axis, and a score region as a region surrounded by lines connecting adjacent plotted dots is formed. The score region may be filled with color, hatching, or the like.

In this manner, in the radar chart coordinates CLA, the axes of the items of the benign (an example of a “first state”) features are arranged on the left side of the origin (an example of a “center of the diagram”), and the axes of the items of the malignant (an example of a “second state”) features are arranged on the right side (an example of the “opposite side” with respect to the center of the diagram) of the origin. In the radar chart coordinates CLA, each axis is set to have a relatively larger value as it moves away from the origin, and the analysis result with a relatively high score for more benign and malignant features is plotted on this axis. Accordingly, the score region of the radar chart coordinates CLA spreads on the left side of the origin in a case where the lesion is benign, and the area on the left side is relatively larger than the area on the right side. On the other hand, the score region spreads on the right side of the origin in a case where the lesion is malignant, and the area on the right side is relatively larger than the area on the left side.

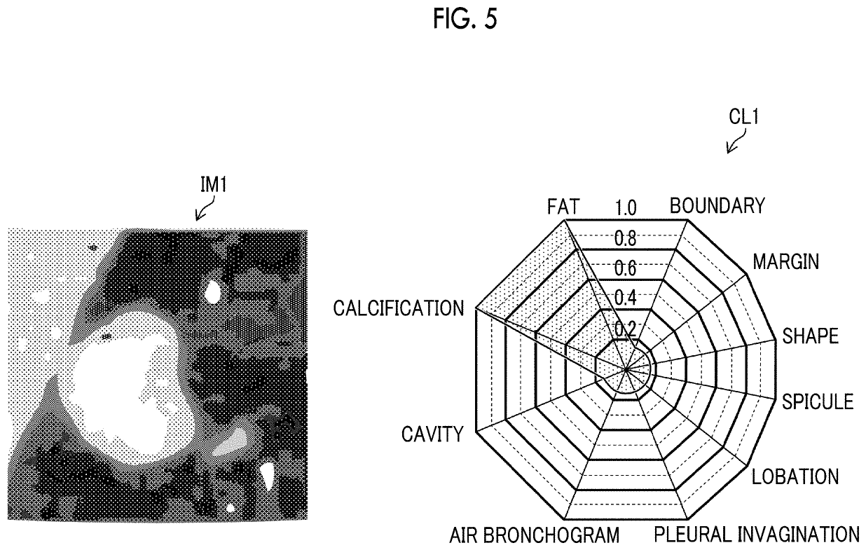

FIG. 5 is a diagram illustrating an example of a medical image IM1 and a radar chart CL1 generated on the basis of the analysis result relating to the characteristics of the lung nodule of the lesion in the medical image IM1. The radar chart CL1 is obtained by plotting the score corresponding to the item of each axis on each axis of the radar chart coordinates CLA. In a case where the score region of the radar chart CL1 is divided left and right sides by a vertical line passing through the origin of the radar chart CL1, the score region spreads to the left side more than the right side, and the area on the left side is relatively larger than the area on the right side. Accordingly, the user can recognize at a glance that the lesion in the medical image IM1 is a benign lung nodule by visually checking the radar chart CL1.

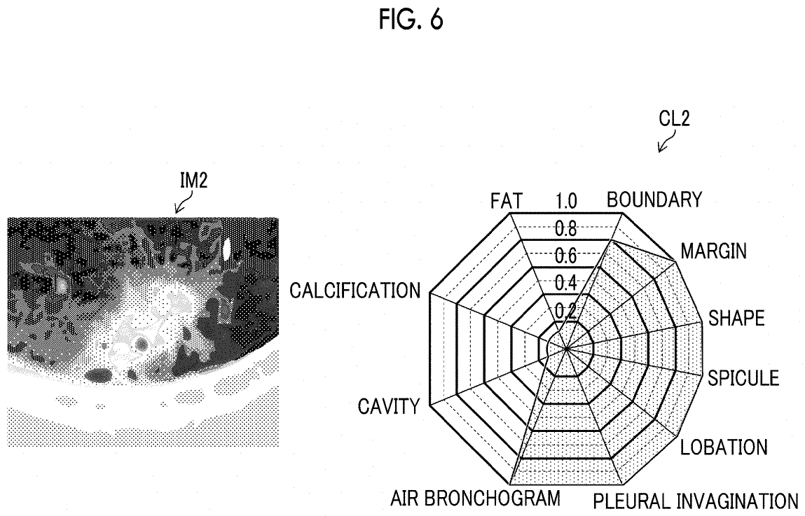

FIG. 6 is a diagram illustrating an example of a medical image IM2 and a radar chart CL2 generated on the basis of the analysis result relating to the characteristics of the lung nodule of the lesion in the medical image IM2. The radar chart CL2 is obtained by plotting the score corresponding to the item of each axis on each axis of the radar chart coordinates CLA. Contrary to the radar chart CL1, in a case where the score region of the radar chart CL2 is divided left and right sides by a vertical line passing through the origin of the radar chart CL2, the score region spreads to the right side more than the left side, and the area on the right side is relatively larger than the area on the left side. Accordingly, the user can recognize at a glance that the lesion in the medical image IM2 is a malignant lung nodule by visually checking the radar chart CL2.

With the radar chart according to the present embodiment, it is possible to express the state of the lesion of the lung nodule having a mutually exclusive relationship by at least one of the spread direction or the area of the score region. Here, the spread direction and the area are expressed by being divided in the left and right sides of the radar chart, but the spread direction and the area are not limited to the left and right sides, and may be expressed by being divided in any direction. Further, the respective axes of the radar chart coordinates may be provided at equal angular intervals.

### Liver Tumor

The radar chart relating to the liver tumor will be described. The state of the liver tumor includes a hepatocellular carcinoma (an example of a “primary cancer”), a metastatic liver cancer (an example of a “metastatic cancer”), and a hemangioma, which have a mutually exclusive relationship. Here, the radar chart will be described which expresses that the larger the area on the right side, the more hepatocellular carcinoma, the larger the area on the lower side, the more metastatic liver cancer, and the larger the area on the left side, the more hemangioma.

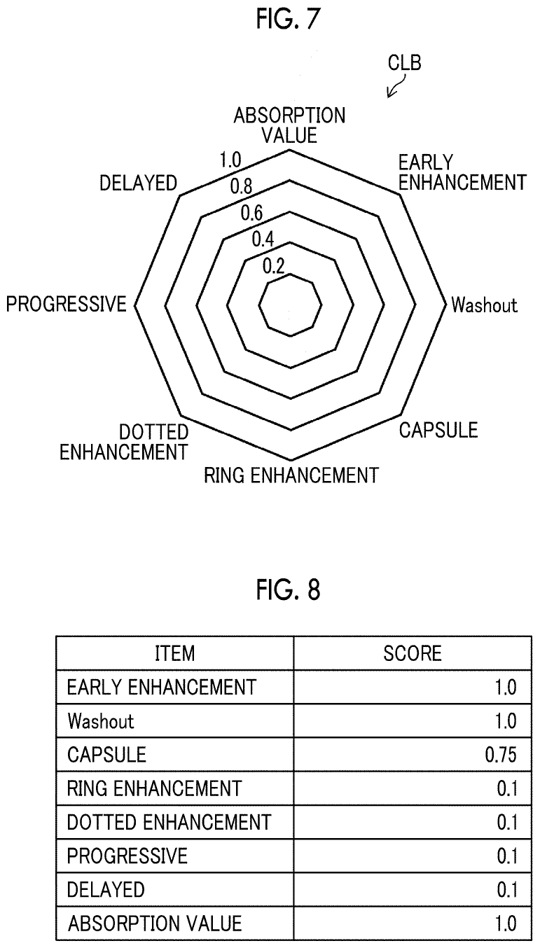

FIG. 7 is a diagram illustrating an example of radar chart coordinates CLB relating to the liver tumor. As illustrated in FIG. 7, the radar chart coordinates CLB has a plurality axes arranged radially around the origin, which respectively correspond to “early enhancement”, “Washout”, “capsule”, “ring-shaped enhancement”, “dotted enhancement”, “progressive”, “delayed” and “absorption value” as eight items relating to the characteristics of the lesion of the liver tumor. The axis of each item is assigned a larger value as it moves away from the origin.

Unlike the radar chart coordinates CLA, in the radar chart coordinates CLB, each axis is not expressed as a line segment, and only scale lines connecting the same values of adjacent axes are expressed as line segments. Further, the items relating to the characteristics of the lesion of the liver tumor are not limited to the eight items described here.

In the radar chart coordinates CLB, the axes of three items of “early enhancement”, “Washout”, and “capsule” which are features of the hepatocellular carcinoma among liver tumors are arranged on the right side of the origin in FIG. 7. In the radar chart coordinates CLB, the axis of the item of “ring-shaped enhancement” which is a feature of the metastatic liver cancer among liver tumors is arranged on the lower side of the origin in FIG. 7. Further, in the radar chart coordinates CLB, the axes of three items of “dotted enhancement”, “progressive”, and “delayed” which are features of the hemangioma among liver tumors are arranged on the left side of the origin in FIG. 7.

In the radar chart coordinates CLB, the axis of the absorption value is arranged on the upper side of the origin in FIG. 7. The absorption value is classified into three categories of “hyperemic”, “anemic”, and “neither”. “Hyperemic” is a feature of “hepatocellular carcinoma”, “anemic” is a feature of “metastatic liver cancer”, and “neither” is a feature of “hemangioma”.

In the radar chart coordinates CLB, scores of each item are plotted on each axis, and a score region surrounded by lines connecting adjacent plotted dots is formed.

In this manner, in the radar chart coordinates CLB, the axes of the items of the features of the hepatocellular carcinoma are arranged on the right side, the axis of the item of the feature of the metastatic liver cancer is arranged on the lower side, the axes of the items of the features of the hemangioma are arranged on the left side and the upper side, and score values are plotted the axis such that a relatively larger value is plotted at a position farther from the origin. Accordingly, the score region of the radar chart coordinates CLB spreads on the right side of the origin in a case where the lesion is a hepatocellular carcinoma, and the area on the right side is relatively larger than the area of the other region. The score region spreads on the lower side of the origin in a case where the lesion is metastatic liver cancer, and the area on the lower side is relatively larger than the area of the other region. The score region spreads on the left side of the origin in a case where the lesion is a hemangioma, and the area on the left side is relatively larger than the area of the other region.

### Hepatocellular Carcinoma

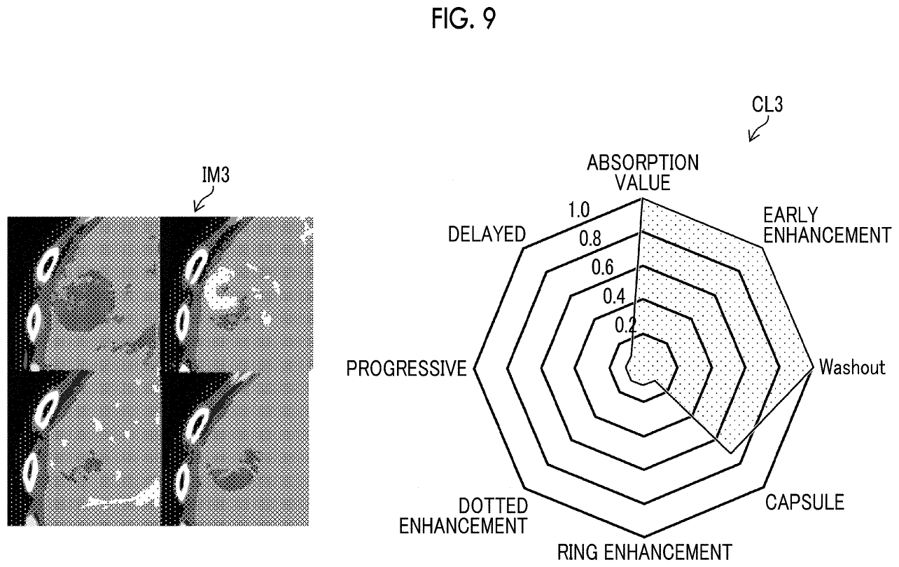

FIG. 8 is a table illustrating an example of a score of each item of the analysis result relating to the characteristics of the liver tumor of a medical image IM3 (refer to FIG. 9).

FIG. 9 is a diagram illustrating an example of the medical image IM3 and a radar chart CL3 generated on the basis of the analysis result relating to the characteristics of the liver tumor of the lesion in the medical image IM3. The radar chart CL3 is obtained by plotting the score corresponding to the item of each axis illustrated in FIG. 8 on each axis of the radar chart coordinates CLB. The score region of the radar chart CL3 spreads on the right side, and the area on the right side is relatively larger than the area of the other region. Accordingly, the user can recognize at a glance that the lesion in the medical image IM3 is a hepatocellular carcinoma by visually checking the radar chart CL3.

### Metastatic Liver Cancer

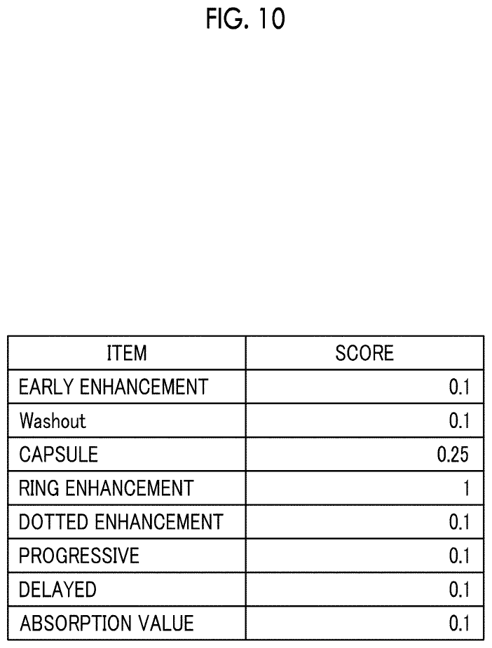

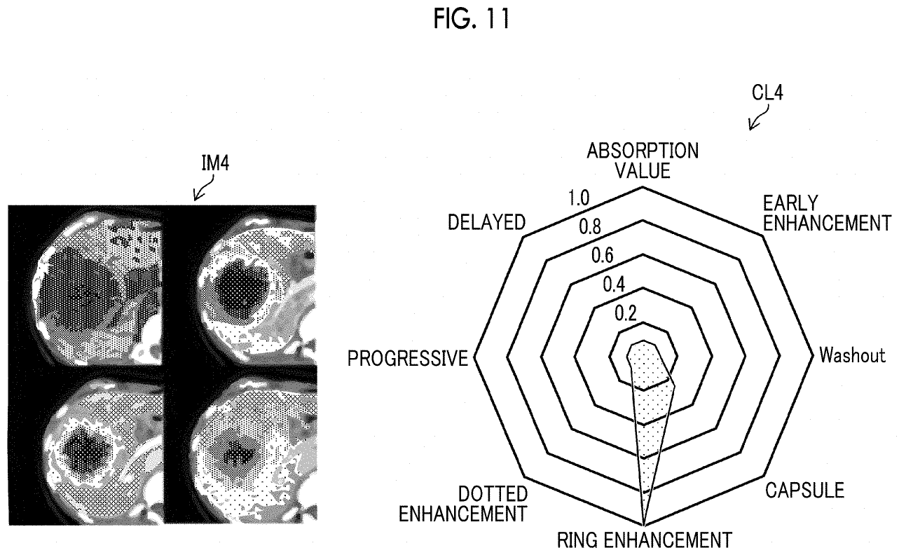

FIG. 10 is a table illustrating an example of a score of each item of the analysis result relating to the characteristics of the liver tumor of a medical image IM4 (refer to FIG. 11).

FIG. 11 is a diagram illustrating an example of the medical image IM4 and a radar chart CL4 generated on the basis of the analysis result relating to the characteristics of the liver tumor of the lesion in the medical image IM4. The radar chart CL4 is obtained by plotting the score corresponding to the item of each axis illustrated in FIG. 10 on each axis of the radar chart coordinates CLB. The score region of the radar chart CL4 spreads on the lower side, and the area on the lower side is relatively larger than the area of the other region. Accordingly, the user can recognize at a glance that the lesion in the medical image IM4 is a metastatic liver cancer by visually checking the radar chart CL4.

### Hemangioma

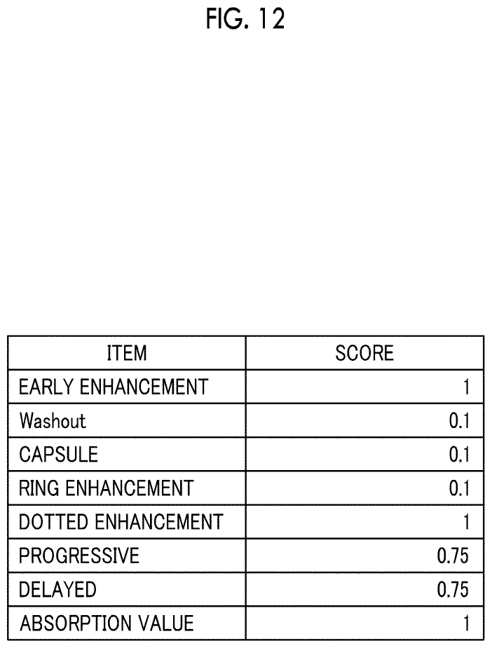

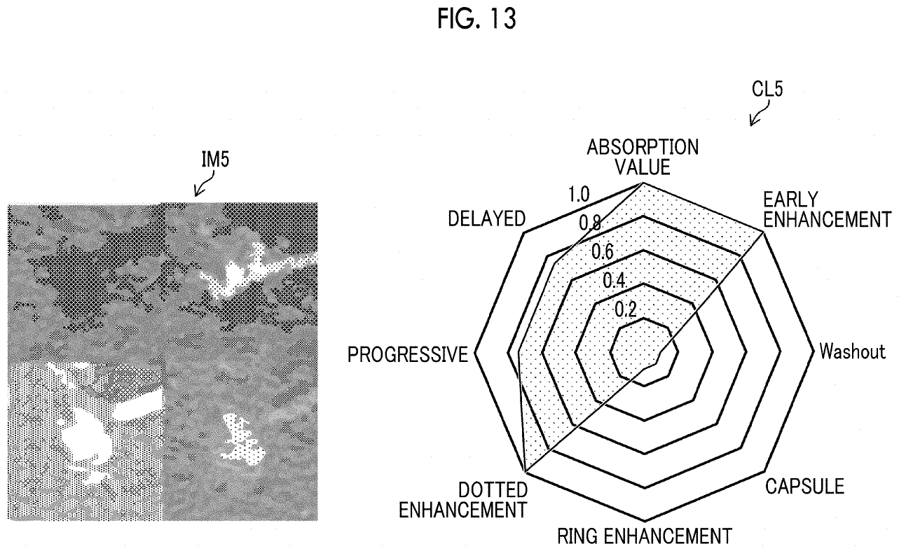

FIG. 12 is a table illustrating an example of a score of each item of the analysis result relating to the characteristics of the liver tumor of a medical image IM5 (refer to FIG. 13).

FIG. 13 is a diagram illustrating an example of the medical image IM5 and a radar chart CL5 generated on the basis of the analysis result relating to the characteristics of the liver tumor of the lesion in the medical image IM5. The radar chart CL5 is obtained by plotting the score corresponding to the item of each axis on each axis of the radar chart coordinates CLB. The score region of the radar chart CL5 spreads on the left side, and the area on the left side is relatively larger than the area of the other region. Accordingly, the user can recognize at a glance that the lesion in the medical image IM5 is a hemangioma by visually checking the radar chart CL5.

With the radar chart according to the present embodiment, it is possible to express the state of the lesion of the liver tumor having a mutually exclusive relationship by at least one of the spread direction or the area of the score region.

### Disease Name

The hepatocellular carcinoma, the metastatic liver cancer, and the hemangioma of the liver tumors have been described as examples in which the state of the region of interest having an exclusive relationship is a disease name. However, the state of the region of interest having an exclusive relationship may be another disease name. That is, for a plurality of disease names that do not occur at the same time, analysis results for a plurality of characteristics may be acquired, and a diagram in which at least one of the spread direction or the area increases the difference in the characteristic features of each disease name may be generated. For example, in a case of the lung nodule, a lung cancer, a hamartoma, an intrapulmonary lymph node, and the like are exemplified as having an exclusive relationship.

### Estimation of State of Region of Interest

The medical information processing apparatus 16 may estimate the state of the lesion. The medical information processing apparatus 16 may estimate the state of the lesion on the basis of at least one of the direction or the area of the diagram.

For example, the medical information processing apparatus 16 can estimate the state of the lesion in the medical image IM1 as “benign” on the basis of the radar chart CL1. Further, the medical information processing apparatus 16 can estimate the state of the lesion in the medical image IM2 as “malignant” on the basis of the radar chart CL2.

The medical information processing apparatus 16 may display the estimated state of the lesion on the display 20B. The medical information processing apparatus 16 may display the estimated state of the lesion in color.

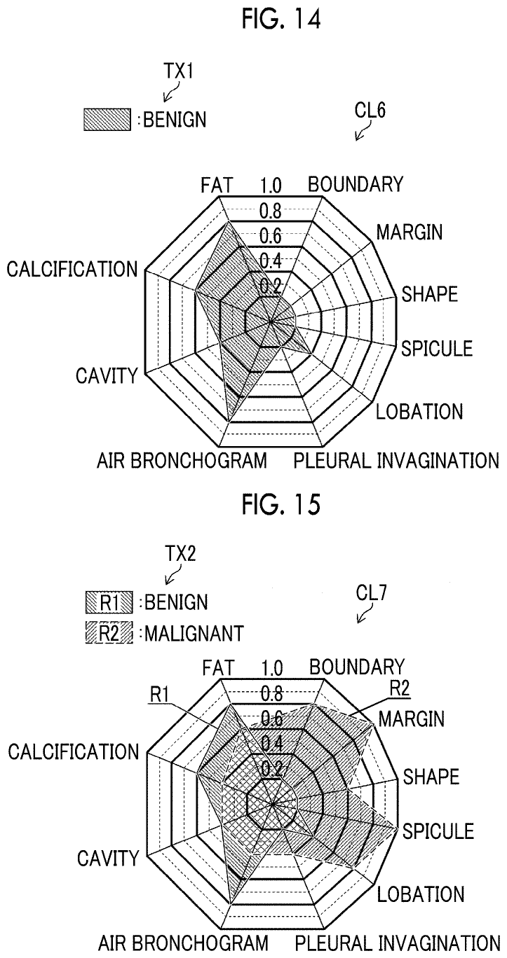

FIG. 14 is a diagram illustrating an example of a radar chart displayed on the display 20B in color. In a radar chart CL6 a score region of the analysis result relating to the characteristics of the lung nodule of the lesion in a medical image (not illustrated) is expressed. In the radar chart CL6, the score region is filled with a blue color in a case where the lesion is determined (estimated) to be “benign”, and the score region is filled with an orange color in a case where the lesion is determined to be “malignant”. Here, the state of the lesion is determined (estimated) to be “benign”, and the score region is filled with a blue color. Further, on the display 20B, “benign” which is the determination result of the state of the lesion is displayed by characters TX1 together with the radar chart CL6.

Note that there may be a plurality of nodules in the same subject. In this case, the analysis result relating to the characteristic of the plurality of lesions may be collectively displayed in one radar chart. FIG. 15 is a diagram illustrating an example of a radar chart which is displayed on the display 20B in color and collectively expresses a plurality of lesions. In a radar chart CL7, score regions of the analysis results relating to the characteristics of the lung nodules of two different lesions in the medical images (not illustrated) are expressed in a superimposed manner. In the radar chart CL7, a first score region R1 for which the state of the lesion is determined to be “benign” is filled with a blue color, and a second score region R2 for which the state of the lesion is determined to be “malignant” is filled with an orange color. Further, on the display 20B, “benign” and “malignant” which are the determination results of the states of the lesions are displayed by characters TX2 together with the radar chart CL7.

In this manner, it is possible for the user to recognize at a glance the state of the lesion of the medical image by displaying the estimated state of the lesion in color.

### Follow-up Examination

In order for the doctor to check the progress of the subject, a follow-up examination is performed on the same subject after a certain period has elapsed in some cases. The medical information processing apparatus 16 may acquire analysis results of a plurality of images of the same subject, the plurality of images being captured at different times, and generates a diagram on the basis of the analysis results of the plurality of images. For example, the medical information processing apparatus 16 may plot the analysis result of the current image, which is the latest medical image, on the diagram coordinates, and may plot the analysis result of the past image, which is the medical image captured in the past, on the same diagram coordinates in a superimposed manner. That is, the analysis results of the plurality of images may be displayed on one diagram.

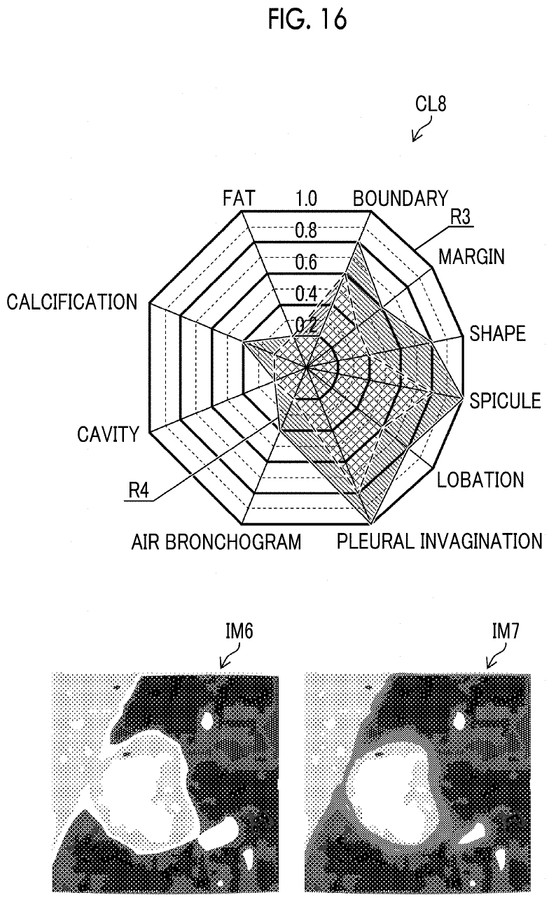

FIG. 16 is a diagram illustrating an example of a latest medical image IM6 and a past medical image IM7 of the same subject, which are a plurality of images captured at different times, and a radar chart CL8 generated on the basis of the analysis results relating to the characteristics of the lesions in the medical image IM6 and the medical image IM7. Similarly to the radar chart coordinates CLA, the radar chart CL8 expresses that the larger the area on the left side, the more benign, and the larger the area on the right side, the more malignant.

Further, in the radar chart CL8, a third score region R3 as the analysis result relating to the characteristics of the lung nodule of the region of interest in the medical image IM6, and a fourth score region R4 as the analysis result relating to the characteristics of the lung nodule of the region of interest in the medical image IM7 are superimposed and displayed in different colors.

In this manner, it is possible for the user to more clearly grasp which item has changed from benign to malignant by displaying the analysis result of the past image on the radar chart indicating the analysis result of the current image in a superimposed manner.

### Different Imaging Conditions and Different Modalities (Imaging Devices)

The medical information processing apparatus 16 may acquire the analysis results of a plurality of images, for which at least one of the imaging condition or the imaging device is different, of the same subject. Further, the different imaging conditions may relate to CT examinations using a contrast medium and contrast conditions for MRI examinations.

### Analysis of Interpretation Report

The analysis result of the region of interest in the medical image may be acquired by analyzing the interpretation report.

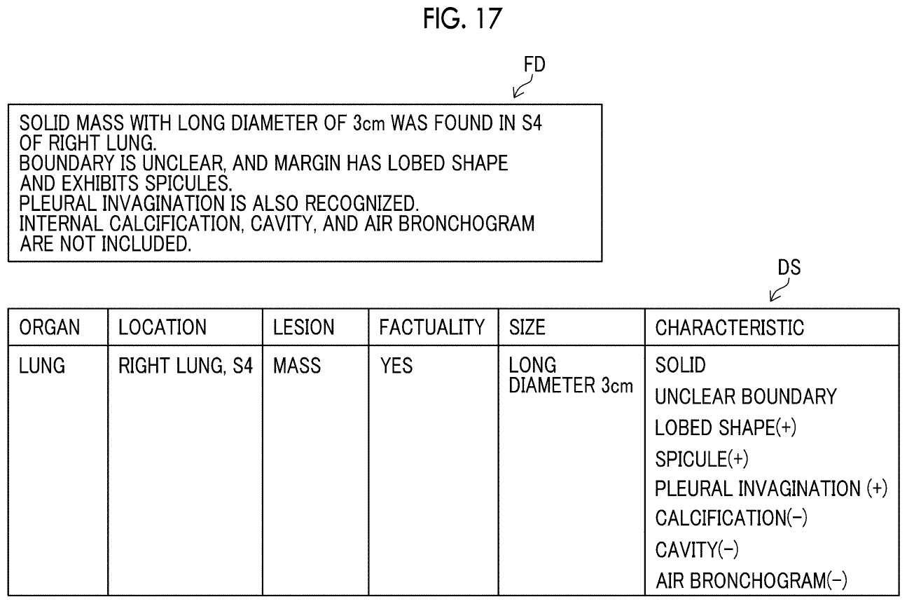

FIG. 17 is a diagram illustrating an example of contents of the interpretation report relating to the medical image (not illustrated), and illustrates a findings sentence FD and structured data DS of the findings sentence FD which are included in the interpretation report. As illustrated in FIG. 17, the findings sentence FD describes “A solid mass with a long diameter of 3 cm was found in S4 of the right lung. The boundary is unclear, and the margin has a lobed shape and exhibits spicules. Pleural invagination is also recognized. Internal calcification, cavity, and air bronchogram are not included”.

The structured data DS is obtained by structuring the findings sentence FD using a natural language technology. The structured data DS has “organ”, “location”, “lesion”, “factuality”, “size”, and “characteristic” as the attributes. Here, the findings sentence FD is structured as “organ” is “lung”, “location” is “right lung, S4”, “lesion” is “mass”, “factuality” is “yes”, “size” is “long diameter of 3 cm”, and “characteristic” is “solid, unclear boundary, lobed shape (+), spicule (+), pleural invagination (+), calcification (−), cavity (−), air bronchogram (−)”.

The medical information processing apparatus 16 acquires the analysis result relating to the characteristics of the region of interest in the medical image from the structured data DS. In this case, as the axis of the radar chart, a previously defined axis may be used, or an axis may be defined focusing on the items of the characteristics described in the interpretation report. Further, in a case of using a previously defined axis, the score may be plotted only for the items of the characteristics described in the interpretation report without plotting the items of the characteristics not described in the interpretation report.

The medical information processing apparatus 16 may generate the radar chart by combining (merging) the analysis result of the medical image and the analysis result of the interpretation report. In a case where the analysis result of the medical image and the analysis result of the interpretation report are different, the analysis result of the interpretation report may be preferentially adopted.

Further, the medical information processing apparatus 16 may generate the radar chart by identifiably distinguishing the analysis result of the medical image from the analysis result of the interpretation report. For example, in a case where “spicule” is described in the interpretation report, the characters of the item of “spicule” of the radar chart may be displayed in a red color, which is a different color from the other items.

In the follow-up examination, in a case where a characteristic, which was not described in the interpretation report of the past image, is newly described in the interpretation report of the current image, the characteristic may be displayed in the radar chart to be identifiably distinguished.

### Interpretation Report

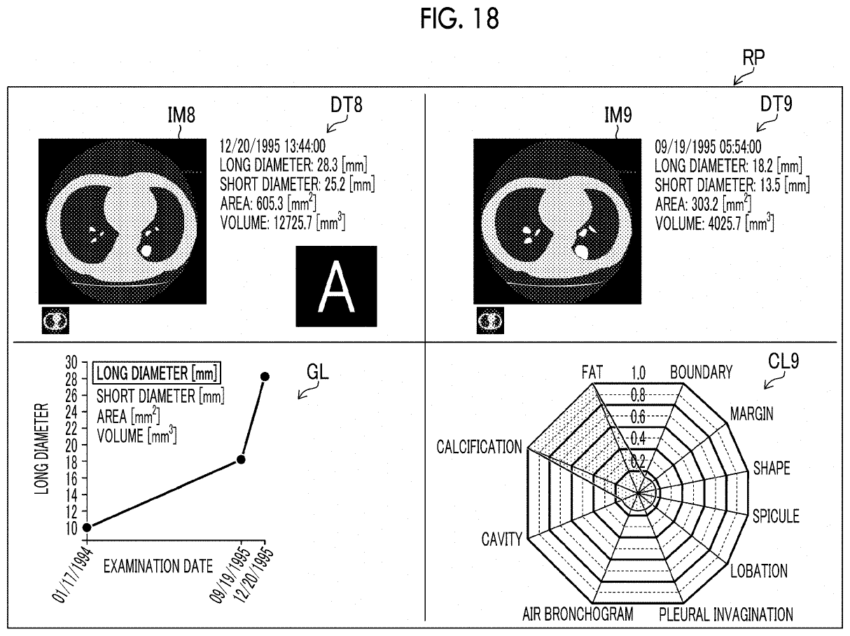

The diagram is attached to the interpretation report. FIG. 18 is a diagram illustrating the interpretation report which is displayed on the display 20B and to which the radar chart is attached. As illustrated in FIG. 18, an interpretation report RP includes a medical image IM8, image information DT8, a medical image IM9, image information DT9, a graph GL, and a radar chart CL9. The interpretation report RP may include a findings sentence.

The medical image IM8 and the image information DT8 are arranged on the upper left side of the interpretation report. The medical image IM8 is the current image of the subject. The image information DT8 is information relating to the medical image IM8. The image information DT8 includes items and respective values of the imaging date and time, and a long diameter [unit: mm], a short diameter [unit: mm], an area [unit: mm²], and a volume [unit: mm³] of the lesion.

The medical image IM9 and the image information DT9 are arranged on the upper right side of the interpretation report. The medical image IM9 is the past image of the same subject. The image information DT9 is information relating to the medical image IM9, and has the same items as in the image information DT8.

The graph GL is arranged on the lower left side of the interpretation report. The graph GL is a polygonal line graph indicating the changes of the lesion. In the graph GL, the user can select any one item of the long diameter [unit: mm], the short diameter [unit: mm], the area [unit: mm²], or the volume [unit: mm³] of the lesion. In the graph GL, the lateral axis is the examination date (imaging date), and the vertical axis is the numerical value of the selected item. In the example illustrated in FIG. 18, “long diameter [mm]” is selected, and the changes of the long diameter of the lesion are displayed.

The radar chart CL9 is arranged on the lower right side of the interpretation report. The radar chart CL9 is generated on the basis of the analysis result relating to the characteristics of the lung nodule of the lesion in the medical image IM8. The axes of the radar chart CL9 are the same as in the radar chart coordinates CLA (refer to FIG. 4).

### Diversion to Non-Medical Image

The diagram according to the present embodiment can be applied to non-medical images. For example, for social infrastructure facilities such as transportation, electricity, gas, and water, an analysis result relating to the characteristics of a region of interest of an image can be acquired, and a diagram expressing the state of the region of interest having an exclusive relationship by at least one of a spread direction or an area can be generated on the basis of the analysis result. The state of the region of interest having an exclusive relationship may be, for example, the necessity of repair.

For example, a region of interest may be set from an image of a road surface of a road, the image of the region of interest may be analyzed, items of characteristics such as elapsed years, depth of damage, size of damage, position of damage (edge or center of road) may be output as a score, and the result may be displayed as a diagram.

### Others

The technical scope of the present invention is not limited to the scope described in the above embodiments. The configurations and the like in the embodiments can be appropriately combined between the embodiments in a range not departing from the gist of the present invention.

### EXPLANATION OF REFERENCES

10: medical information processing system

12: medical image examination device

14: medical image database

16: medical information processing apparatus

16A: processor

16B: memory

16C: communication interface

18: interpretation report database

20: user terminal

20A: input device

20B: display

22: network

CL1: radar chart

CL2: radar chart

CL3: radar chart

CL4: radar chart

CLS: radar chart

CL6: radar chart

CL7: radar chart

CL8: radar chart

CL9: radar chart

CLA: radar chart coordinates

CLB: radar chart coordinates

DS: structured data

DT8: image information

DT9: image information

FD: findings sentence

GL: graph

IM1: medical image

IM2: medical image

IM3: medical image

IM4: medical image

IM5: medical image

IM6: medical image

IM7: medical image

IM8: medical image

IM9: medical image

R1: first score region

R2: second score region

R3: third score region

R4: fourth score region

RP: interpretation report

ST1-ST5: step of medical information processing method

TX1: characters

TX2: characters

What is claimed is:

1. An information processing apparatus comprising:
at least one processor; and
at least one memory that stores a command for the at least one processor to execute,
wherein the at least one processor is configured to
acquire an analysis result relating to a characteristic of a region of interest of an image, and
generate a diagram on the basis of the analysis result, and
the diagram expresses a state of the region of interest having an exclusive relationship by at least one of a spread direction or an area.

2. The information processing apparatus according to claim 1,
wherein the image is a medical image in which a subject is imaged.

3. The information processing apparatus according to claim 2,
wherein the state of the region of interest is benign and malignant.

4. The information processing apparatus according to claim 2,
wherein the state of the region of interest is a primary cancer and a metastatic cancer.

5. The information processing apparatus according to claim 2,
wherein the state of the region of interest is a disease name.

6. The information processing apparatus according to claim 1,
wherein the at least one processor displays the diagram on a display.

7. The information processing apparatus according to claim 1,
wherein the at least one processor is configured to
estimate the state of the region of interest, and
display an estimated result on a display.

8. The information processing apparatus according to claim 7,
wherein the at least one processor estimates the state of the region of interest on the basis of at least one of the direction or the area of the diagram.

9. The information processing apparatus according to claim 1,
wherein the diagram includes a radar chart.

10. The information processing apparatus according to claim 1,
wherein the at least one processor acquires the analysis results of a plurality of images of the same subject, the plurality of images being captured at different times.

11. The information processing apparatus according to claim 10,
wherein the diagram expresses the analysis results of the plurality of images in a superimposed manner.

12. The information processing apparatus according to claim 11,
wherein the diagram expresses the analysis results of the plurality of images in different colors.

13. The information processing apparatus according to claim 1,
wherein the at least one processor acquires the analysis results of a plurality of images, for which at least one of an imaging condition or an imaging device is different, of the same subject.

14. The information processing apparatus according to claim 13,
wherein the imaging condition includes a contrast condition.

15. The information processing apparatus according to claim 1,
wherein the at least one processor acquires the analysis result by analyzing the image.

16. The information processing apparatus according to claim 1,
wherein the at least one processor acquires the analysis result by analyzing an interpretation report of the image.

17. The information processing apparatus according to claim 16,
wherein the at least one processor generates at least one of: a diagram in which the analysis result acquired by analyzing the interpretation report and the analysis result acquired by analyzing the image are combined; and a diagram in which the analysis result acquired by analyzing the interpretation report and the analysis result acquired by analyzing the image are distinguished from each other.

18. An information processing system comprising:
the information processing apparatus according to claim 1;
an imaging device that captures the image; and
a display on which the diagram is displayed.

19. An information processing method comprising:
an analysis result acquisition step of acquiring an analysis result relating to a characteristic of a region of interest of an image; and
a diagram generation step of generating a diagram on the basis of the analysis result,
wherein the diagram expresses a state of the region of interest having an exclusive relationship by at least one of a spread direction or an area.

20. A non-transitory, computer-readable tangible recording medium which records thereon, a program for causing, when read by a computer, the computer to execute the information processing method according to claim 19.
